(19) United States
(12) Patent Application Publication
(10) Pub. No.: US 2012/0197313 A1
(43) Pub. Date: Aug. 2, 2012

(54) Angled Receiver Wall for Provisionally Fixing a Crown in a Multi-Axial Screw Assembly

(75) Inventor: Benjamin Cowan, Memphis, TN (US)

(73) Assignee: WARSAW ORTHOPEDIC, INC., Warsaw, IN (US)

(21) Appl. No.: 13/016,534

(22) Filed: Jan. 28, 2011

(51) Int. Cl. A61B 17/86; B23P 11/02

(52) U.S. Cl. 606/305; 29/453

(57) ABSTRACT

A multi-axial screw assembly comprises a bone anchoring member, a receiver, a crown and a snap ring. The receiver member has an aperture extending along a central axis of the receiver member. The aperture receives the bone anchoring member. The receiver member has a cutout on an interior wall of the receiver member. The cutout has an angled wall surface. The angled wall surface extends along the central axis of the receiver. The crown is configured to be received within the receiver member along the central axis of the receiver member. The crown has a groove around the circumference of the crown. The snap ring is configured to be received in the groove of the crown and in the cutout in the receiver member. The snap ring exerts an axial force on the crown when the snap ring abuts the angled wall surface of the receiver member.

### FIELD OF INVENTION

Embodiments of the invention relate to implants used for correction of orthopedic injuries or deformities, and more specifically, but not exclusively, relate to multi-axial screws implanted in bone for stabilizing longitudinal support members.

### BACKGROUND

Typical implant systems include several pieces, which may be associated or useful with only specific other pieces. Among such pieces are screws, hooks rods, plates and similar longitudinal members for supporting, holding and/or correcting one or more bones. Such longitudinal members can be fastened to bones via direct or indirect connection to hooks, screws, bolts or other fasteners, and may be linked to each other by a variety of connectors. In the spinal field, for example, screws or other fasteners can be attached to two or more vertebrae, the vertebrae can be adjusted into their normal or a therapeutically better position, and longitudinal members are connected to the fasteners so that the vertebrae are held in the normal or therapeutically improved position.

Accordingly, known bone screws, hooks, clamps and other bone fasteners or fixation devices can be connected or adjoined to a particular bone or bones as a connection between the remainder of the implant and the bone(s). Where a rod is used as a support and stabilizing member, commonly a series of two or more screws are inserted into two or more vertebrae to be instrumented. A rod is then placed within or coupled to the heads of the screws, or is placed within a connecting device that links the rod and a screw head, and the connections are tightened. In this way, a rigid supporting structure is fixed to the vertebrae, with the rod providing the support that maintains and/or promotes correction of the vertebral malformation or injury.

Some devices allow one or more degrees of freedom between a fastening portion or fastening member and a receiving portion or member, reducing the required precision of placement of the fixation device, since a head portion of the fixation device is multi-axially positionable around the bone-threaded or hook portion. The head can thus be positioned so as to easily receive the rod, limiting or removing much of the positioning difficulty inherent in prior devices. However, such multi-angle positioning between the fastening portion and the receiving portion for every relative orientation of those parts may create difficulty in orienting the parts during surgery.

The description herein of problems and disadvantages of known apparatuses, methods, and devices is not intended to limit the invention to the exclusion of these known entities. Indeed, embodiments of the invention may include, as a part of the embodiment, portions or all of one or more of the known apparatus, methods, and devices without suffering from the disadvantages and problems noted herein.

### SUMMARY OF THE INVENTION

An aspect of the invention may include a multi-axial screw assembly comprising a bone anchoring member, a receiver, a crown and a snap ring. The receiver member has an aperture extending along a central axis of the receiver member. The aperture receives the bone anchoring member. The receiver member has a cutout on an interior wall of the receiver member. The cutout has an angled wall surface. The angled wall surface extends along the central axis of the receiver. The crown is configured to be received within the receiver member along the central axis of the receiver member. The crown has a groove around the circumference of the crown. The snap ring is configured to be received in the groove of the crown and in the cutout in the receiver member. The snap ring exerts an axial force on the crown when the snap ring abuts the angled wall surface of the receiver member.

Another aspect of the invention may include a multi-axial screw assembly comprising a bone anchoring member, a receiver, a crown and a snap ring. The receiver member has an aperture extending along a central axis of the receiver member. The aperture receives the bone anchoring member. The receiver member has a cutout on an interior wall of the receiver member. The crown is configured to be received within the receiver member along the central axis of the receiver member. The crown has a groove on an outer surface of the crown. The groove has an angled wall surface. The angled wall surface extends along the central axis of the receiver. The snap ring is configured to be received in the groove of the crown and in the cutout in the receiver member. The snap ring exerts an axial force on the crown when the snap ring abuts the angled wall surface of the receiver member.

Yet another aspect of the invention provides a method of provisionally fixing a bone anchoring member to a receiver member in a multi axial screw assembly. The method includes the step of loading a bone anchoring member in a receiver member. Another step compresses a snap ring between a crown and a receiver member. A positioning step positions the crown and snap ring within the receiver member such that the snap ring expands into a cutout in the receiver member. The snap ring abuts an angled wall surface thereby forcing the crown against the bone anchoring member.

Additional aspects and features of the present disclosure will be apparent from the detailed description and claims as set forth below.

### BRIEF DESCRIPTION OF THE DRAWINGS

FIG. 1 is a cross sectional view of a multi-axial screw according to an aspect of the invention.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

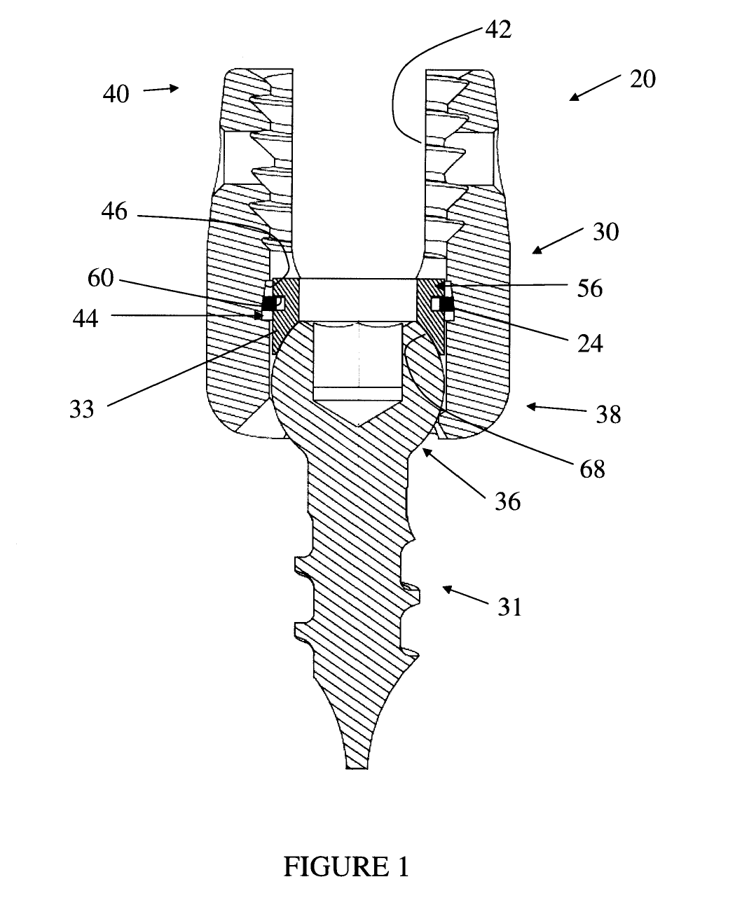

FIG. 1 is a cross sectional view of a multi-axial screw according to an aspect of the invention. Multi-axial screw assembly 20 includes a snap ring 24, a receiver member 30, a bone anchoring member 31, and a crown 33. The assembly 20 is configured such that the bone anchoring member 31 is captured between the receiver member 30 and the crown 33 along a central axis of the assembly 20. Receiver member 30 is also configured to accommodate a rod or other longitudinal member through an aperture generally perpendicular to the bone anchoring member 31. In a specific embodiment, the receiver member 30 includes a lower portion 38 and a upper portion 40, a threaded portion 42 at or near upper portion 40 receives a compression member (for example, a set screw or other element with external threads) to capture the rod within the receiver member 30. Threaded portion 42 could be outside of receiver member 30 if an external compression member is used. Alternatively, receiver member 30 could be externally and/or internally configured for compression members using snapping, twisting or other types of closures. The lower portion 38 of receiver member 30 has a cutout 44. In the illustrated embodiment, cutout 44 extends around an aperture in the receiver member 30 around the central axis.

Snap ring 24 is received between the crown 33 and the receiver member 30. The snap ring axially fixes the crown 33 within the receiver member 30 while allowing relative rotation between these parts 30 and 33. The snap ring 24 is received under a flange 56 of the crown 33. The snap ring 24, when it expands, sits under the flange 56 in the cutout 44 inside the receiver member 30.

The bone anchoring member 31 includes a head portion 36. The head portion 36 rests in the lower portion 38 of the receiver member 30. The crown 33 may contact the head portion 36 of the bone anchoring member 31. As the force of the contact of the head portion 36 with the crown 33 increases (as the head portion 36 is pressed upon the lower portion 38 of the receiver 30) the bone anchoring member 31 is provisionally held in place relative to the receiver member 33. As explained below, the amount of force holding the crown 33 down against the head portion 36 of the bone anchoring member 31 may be controlled by the snap ring 24 abutting the surface of the cutout 44 in the receiver member 30.

The cutout 44 in the receiver member 30 is configured with an angled wall portion 46. The cutout 44 extends vertically along the central axis of the receiver member for a distance greater than the thickness of the snap ring 24. The cutout 44 may extend entirely around the circumference of the receiver member 33. Similarly, the angled wall portion 46 may extend around the entire circumference of the receiver member 30. Alternatively, either the cutout 44 or the angled wall portion 46 may extend around only a portion of the receiver member 30, which would then cause the snap ring to asymmetrically sit along the central axis of the receiver member 30.

The angled wall portion 46 varies the diameter of the cutout 44 from a first, larger diameter nearer the bottom of the receiver member 30 to a second, smaller diameter nearer the top of the receiver 30. The smaller diameter portion of the angled wall portion 46 forces the snap ring 24 to radially compress. The radial compression in the snap ring 24 against the angled wall portion 46 exerts axial forces on the crown 33. The crown 33, then, is pressed down on the head portion 36 of the bone anchoring member 31. As the crown 33 is pushed axial toward the upper portion 40 of the receiver member 30, the radial compression of the snap ring 24 continues to increase as the angled wall portion 46 continues to become smaller in diameter. This increases the downward axial force against the crown 33 and thus the friction between the head portion 36 of the bone anchoring member 31 and the receiver member 30. While the angled wall portion 46 has been shown as part of the receiver member 30, in another embodiment, the angled wall portion may be part of the crown. In such an embodiment, the angled surface in the crown would transfer force between the compressed snap ring and the crown as the snap ring is compressed against the receiver member.

Crown 33 is sized to fit within receiver member 30 so that crown 33 has some freedom of axial movement within the receiver member 30. An undersurface 68 is preferably configured to accommodate at least a part of head portion 36 of bone anchoring member 31. For example, undersurface 68 may be shaped (e.g. spherical, rounded, conical, or otherwise) to allow relative movement between crown 33 and part or all of head portion 36 of bone anchoring member 31. In the embodiment in which both undersurface 68 and head portion 36 have a rounded or spherical portion, undersurface 68 may have substantially the same or greater diameter as head portion 36.

The crown 33 also has a groove 60. The groove 60 receives the snap ring 24 when the snap ring 24 and crown 33 are inserted into the receiver member 30. Because the snap ring 24 is at least partially retained in the groove 68 of the crown 33, the snap ring 33 is capable of transferring the axial force to the crown 33 created through the radial compression of the snap ring 24 against the angled wall portion 46.

The foregoing detailed description is provided to describe the invention in detail, and is not intended to limit the invention. Those skilled in the art will appreciate that various modifications may be made to the invention without departing significantly from the spirit and scope thereof.

Furthermore, it is understood that all spatial references, such as “first,” “second,” “exterior,” “interior,” “superior,” “inferior,” “anterior,” “posterior,” “central,” “annular,” “outer,” and “inner,” are for illustrative purposes only and can be varied within the scope of the disclosure.

1. A multi axial screw assembly, comprising:
a) a bone anchoring member;
b) a receiver member having a channel configured to receive a rod and an aperture extending along a central axis of the receiver member, the aperture configured to receive the bone anchoring member, the receiver member having a cutout on an interior wall of the receiver member, the cutout having an angled wall surface, the angled wall surface extending along the central axis of the receiver;
c) a crown configured to be received within the receiver member along the central axis of the receiver member, the crown having a groove on an outer surface of the crown; and
d) a snap ring configured to be received in the groove of the crown and in the cutout in the receiver member, wherein the snap ring exerts an axial force on the crown when the snap ring abuts the angled wall surface of the receiver member.

2. The multi axial screw assembly of claim 1, wherein the snap ring has a thickness, the angled wall surface extends along the interior wall of the receiver member for a distance greater than the thickness of the snap ring.

3. The multi axial screw assembly of claim 1, wherein the crown has an undersurface and the bone anchoring member has a head portion, the undersurface of the crown having a radius of curvature generally equal to a radius of curvature of the head portion of the bone anchoring member.

4. The multi axial screw assembly of claim 1, wherein the cutout extends around the circumference of the interior wall of the receiver member.

5. The multi axial screw assembly of claim 1, wherein the angled wall surface extends around the circumference of the interior wall of the receiver member.

6. A multi axial screw assembly, comprising:
a) a bone anchoring member;
b) a receiver member having a channel configured to receive a rod and an aperture extending along a central axis of the receiver member, the aperture configured to receive the bone anchoring member, the receiver member having a cutout on an interior wall of the receiver member;
c) a crown configured to be received within the receiver member along the central axis of the receiver member, the crown having a groove on an outer surface of the crown, the groove having an angled wall surface, the angled wall surface extending along the central axis of the receiver; and
d) a snap ring configured to be received in the groove of the crown and in the cutout in the receiver member, wherein the snap ring exerts an axial force on the crown when the snap ring abuts the angled wall surface of the receiver member.

7. The multi axial screw assembly of claim 6, wherein the snap ring has a thickness, the angled wall surface extends along the outer surface of the crown for a distance greater than the thickness of the snap ring.

8. The multi axial screw assembly of claim 1, wherein the crown has an undersurface and the bone anchoring member has a head portion, the undersurface of the crown having a radius of curvature generally equal to a radius of curvature of the head portion of the bone anchoring member.

9. The multi axial screw assembly of claim 1, wherein the cutout extends around the circumference of the interior wall of the receiver member.

10. The multi axial screw assembly of claim 1, wherein the angled wall surface extends around the circumference of the outer surface of the crown.

11. A method of provisionally fixing a bone anchoring member to a receiver member in a multi axial screw assembly, comprising the steps of:
a) loading a bone anchoring member in a receiver member;
b) compressing a snap ring between a crown and a receiver member; and
c) positioning the crown and snap ring within the receiver member such that the snap ring expands into a cutout in the receiver member, the snap ring abutting an angled wall surface thereby forcing the crown against the bone anchoring member.
